(19) United States
(12) Plant Patent Application Publication
(10) Pub. No.: US 2020/0323116 P1
(43) Pub. Date: Oct. 8, 2020

(54) Fragaria L. plant named 'Tochigi iW1 GO'

(50) ;

(71) Applicant: TOCHIGI PREFECTURE, Utsunomiya-shi (JP)

(72) Inventors: Tatsuro NAKANISHI, Utsunomiya-shi (JP); Risa TSURUMI, Utsunomiya-shi (JP); Takashi OHASHI, Utsunomiya-shi (JP); Yoshiyuki ISHIHARA, Utsunomiya-shi (JP)

(21) Appl. No.: 16/501,393

(22) Filed: Apr. 5, 2019

(51) Int. Cl. A01H 6/74

(52) U.S. Cl. PLT/208

(57) ABSTRACT

‘Tochigi iW1 Go’ is a new variety of strawberry bred by crossbreeding variety ‘Wadahatsukoi’ and ‘09-52-1’ in 2012. The plant may be used, e.g., for cultivation of fruit for consumption. ‘Tochigi iW1 Go’ is a high-quality, high-yielding variety that produces a fruit having a white pericarp, a large fruit size, a high-yielding characteristic, and adaptability to forcing culture.

### PLANT NAME AND VARIETY DENOMINATION

Latin name of the genus and species of the plant claimed: Fragaria L.

Variety denomination: ‘Tochigi iW1 Go’.

### BACKGROUND

Fragaria is a genus of flowering plants in the Rosaceae family, commonly known as strawberries for their edible fruits. Strawberries have a taste that varies by cultivar, and ranges from quite sweet to rather tart. Strawberries are an important commercial fruit crop, widely grown in all temperate regions of the world.

Strawberries are cultivated worldwide for their fruit. The fruit is widely known for its characteristic aroma, bright red color, juicy texture, and sweetness. The strawberry is not, from a botanical point of view, a berry. Technically, it is an aggregate accessory fruit, meaning that the fleshy part is derived not from the plant's ovaries but from the receptacle that holds the ovaries.

Propagation is often by runners, which can be pegged down to encourage them to take root, or cut off and placed in a new location. Strawberries are popular plants to grow in the domestic environment, for consumption or exhibition purposes, almost anywhere in the world.

### BRIEF SUMMARY

‘Tochigi iW1 Go’ is a new variety of Fragaria (F. x ananassa) bred by crossbreeding seedling series of variety ‘Wadahatsukoi’ as the female parent and variety ‘09-52-1’ (not patented) as the male parent in 2012. In 2013, a ‘13-w1-2’ line was selected from among the crossbreeds and named ‘Tochigi iW1 Go’. The plant may be used, e.g., for cultivation of fruit for consumption.

‘Tochigi iW1 Go’ is a high-quality, high-yielding variety that produces a fruit having a white pericarp, a taste and a quality similar to those of ‘Tochiotome’ (not patented), a large fruit size, a high-yielding characteristic, and adaptability to forcing culture. This line is a non-remontant strawberry for forcing culture, having been bred in development of varieties suitable for processing use, and has a characteristic that its fruit skin is yellowish-white.

‘WADAHATSUKOI’ is a Frageria cultivar having medium to large fruit with a pinkish-white color.

‘Tochigi iW1 Go’ was asexually reproduced via runners in Tochigi-shi, Tochigi Japan.

### BRIEF DESCRIPTION OF THE DRAWINGS

FIG. 1 is a photograph of a ‘Tochigi iW1 Go’ plant body at less than one year of age.

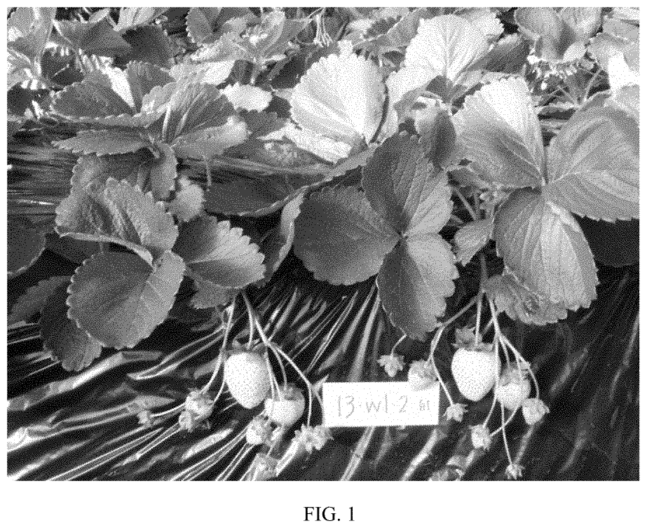

FIG. 2 is a photograph of the fruit of a ‘Tochigi iW1 Go’ plant (left) and the fruit of a ‘Tochiotome’ plant (right) at less than one year of age.

FIG. 3 is a photograph of a cross-sectional view of the fruit of a ‘Tochigi iW1 Go’ plant (left) and a cross-sectional view of the fruit of a ‘Tochiotome’ plant (right) at less than one year of age.

FIG. 4 is a photograph of the fruit of a ‘Tochigi Shiro 2 Go’ plant (left), the fruit of a ‘Wadahatsukoi’ plant (center), and the fruit of a ‘Tochigi iW1 Go’ plant (right) at less than one year of age.

FIG. 5 is a photograph of the fruit of a ‘Tochigi iW1 Go’ plant at less than one year of age.

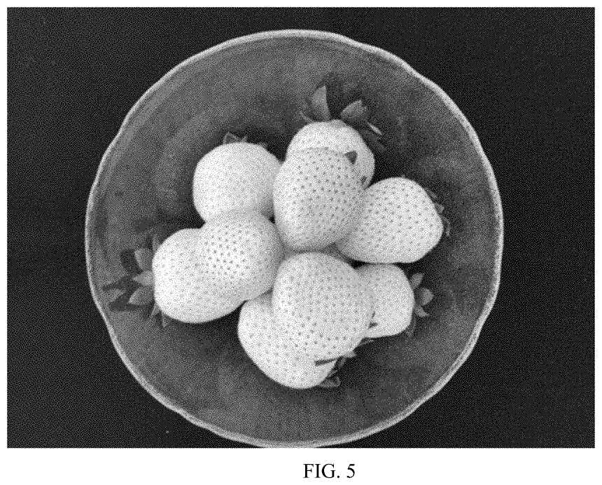

FIG. 6 is a photograph of the top surface of the leaf of a ‘Tochigi iW1 Go’ plant at less than one year of age.

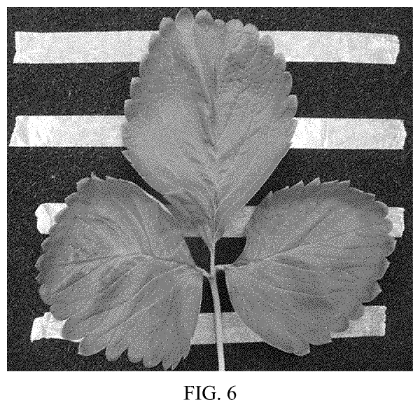

FIG. 7 is a photograph of the bottom surface of the leaf of a ‘Tochigi iW1 Go’ plant at less than one year of age.

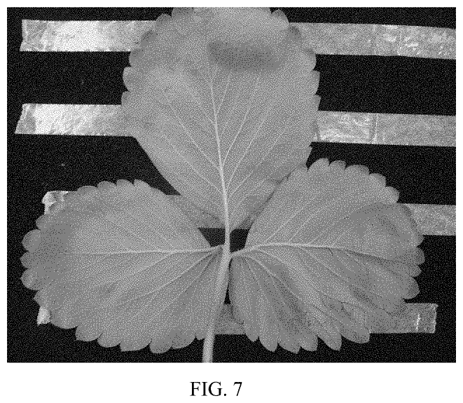

FIG. 8 is a photograph of the flowers of a ‘Tochigi iW1 Go’ plant at less than one year of age.

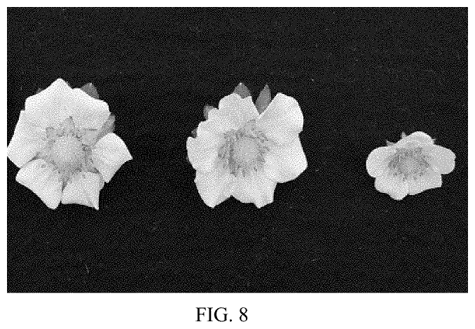

### DETAILED BOTANICAL DESCRIPTION OF THE PLANT

Variety: ‘Tochigi iW1 Go’

Species of the plant claimed: Fragaris L. The claimed plant is bred by crossbreeding seedling series of variety ‘Wadahatsukoi’ as the female parent and variety ‘09-52-1’ (not patented) as the male parent. The parental lines of the claimed plant resulted from the hybridization of different varieties. The parental lines of ‘09-52-1’ are as follows: ‘Tochigi 26 go’ as the female parent and ‘98-7-3’ as the male parent. The parental lines of ‘Wadahatsukoi’ are unknown.

Common names of the claimed plant's species include: Strawberry.

Characteristics of ‘Tochigi iW1 Go’ provided herein were observed when the plant was cultivated at Tochigi-shi, Tochigi, Japan in a simple vinyl greenhouse from 2012 to 2017. Descriptive terms used in Tables 1, 2, and 3 are consistent with the Japanese Examination Standard for Fragaria L., the entire contents of which are incorporated herein by reference. ‘Tochigi iW1 Go’ as described herein was crossbred around March and fixedly planted in September of the same year, and firstly selected around March three years later. The basic body at that time (basic line) was seven years old.

Properties and characteristics of ‘Tochigi iW1 Go’ are described in Table 1. Colors are described using The Royal Horticultural Society (R.H.S.) color chart.

TABLE 1
Properties and characteristics of ‘Tochigi iW1 Go’

| Description | State of Expression |
|---|---|
| Plant: growth habit | spreading |
| Petal: color of upper side | RHS 2901 (yellowish |
| | white) |
| Fruit: size | large to very large; |
| | avg. weight per fruit |
| | 20 g. |
| Fruit: shape | conical |
| Fruit (fruit skin): color | RHS 2701 (yellowish |
| | white) |
| Fruit: color of flesh | RHS 2501 (yellowish |
| (excluding core) | white) |
| Type of bearing | not remontant (one- |
| | season bearing) |

When grown in Tochigi-shi, Tochigi, Japan (or in an area with a similar latitude and climate) ‘Tochigi iW1 Go’ should be planted in mid-September. In this region, flowering time is from late October to early February. In this region, harvesting time is from early December to late March. The claimed variety ‘Tochigi iW1 Go’ can be stored for six days at a temperature of 5 degrees Celsius or lower.

Characteristic pretests were performed in 2015 and 2016 using 10 stocks per sector and a 2 sector system. Research items were growth, yield, and fruit quality. The test farm field used was a pipe house. Planting pattern used was soil culture, two high ridges with ridge width of 100 cm and distance between stocks of 24 cm. Start of heat retention was at the end of October. Temperature control was performed according to the following pattern: a morning temperature of 25° C., afternoon temperature of 23° C., and lowest night temperature of 8° C. from the end of October to mid-December; followed by a morning temperature of 27° C., afternoon temperature of 23° C., and lowest night temperature of 8° C. until late February; and finally a morning temperature of 25° C., afternoon temperature of 23° C., lowest night temperature of 8° C. after late February.

In 2015, forcing culture and cell nursing were used as cropping type and nursing method, and time of fix planting was late September. Base fertilizer content was 1.0 kg/a nitrogen, 1.2 kg/a phosphoric acid, and 0.8 kg/a potash. Additional fertilizer was used, with a content per one fertilization of 0.1 kg/a nitrogen, 0.04 kg/a phosphoric acid, and 0.06 kg/a potash. Additional fertilization was made basically every 14 days from late December until the end of culture.

In 2016, forcing culture, cell nursing, and night cooling nursing were used as cropping type and nursing method, and time of fix planting was early September for night cooling nursing and late September for cell nursing. Base fertilizer content was 2.0 kg/a nitrogen, 2.4 kg/a phosphoric acid, and 1.6 kg/a potash. No additional fertilizer was used.

The growth habit of ‘Tochigi iW1 Go’ is spreading, with a plant height of approximately 20 cm. Plant width has not yet been obtained. The leaf shape of ‘Tochigi iW1 Go’ is odd pinnate, with a dentate margin and reticulate venation pattern. The vein color of ‘Tochigi iW1 Go’ is 9Y2910 (dull greenish yellow). Leaf shape is circular and length is equal in relation to width. Blistering on the surface of the leaf is medium, and glossiness is medium. Leaf blade length of ‘Tochigi iW1 Go’ is 10-11 mm in November and 7-10 mm in January. Leaf width is 9-10 mm in November and approximately 6 mm in January (based on 2017 Research Report). Leaf color is green on both the upper and lower leaf surfaces. Sepal color of ‘Tochigi iW1 Go’ is 5GY3513 (dull yellow green), with a normal texture. Average sepal length and width measurements have not been obtained. Petal length and width, and pedicle and peduncle length, of ‘Tochigi iW1 Go’ differ depending on inflorescence and time. Pedicle and peduncle color is 3GY3312 (dull yellow green). Petal texture is normal, margin is smooth, and color is RHS 2901 (yellowish white). Average number of flowers per panicle for ‘Tochigi iW1 Go’ is 11-12. Ovary color is 7Y2704 (bright greenish yellow) with a conical shape, while anther color is 5Y2507 (vivid yellow) with a long heart shape. Measurements of ovary and anther size have not been obtained.

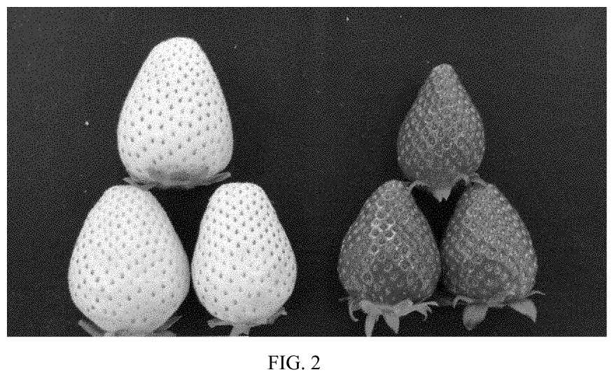

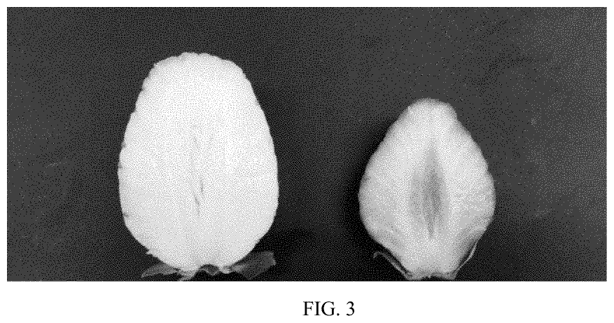

A variety that is similar to ‘Tochigi iW1 Go’ is ‘Tochiotome’. Characteristics of ‘Tochigi iW1 Go’ and ‘Tochiotome’ are compared in Table 2. Fruit of ‘Tochiotome’ are shown in FIGS. 2 and 3.

TABLE 2
Distinguishable characteristics between ‘Tochigi iW1 Go’ and ‘Tochitome’

| Denomination | | State of | State of Expression |
|---|---|---|---|
| of | Description of | Expression in | in |
| Similar Variety | Characteristics | Similar Variety | ‘Tochigi iW1 Go’ |
| TOCHIOTOME | Plant: growth habit | semi-upright | spreading |
| TOCHIOTOME | Fruit: size | large | large to very large |
| TOCHIOTOME | Fruit (fruit skin): | medium red | whitish yellow |
| | color | | |
| TOCHIOTOME | Fruit: color of flesh | light red | whitish |
| | (excluding core) | | |
| TOCHIOTOME | Fruit: color of core | medium red | white |

Growth habit of ‘Tochigi iW1 Go’ is spreading. Vigor is similar to that of ‘Tochiotome’, and gets a bit weaker in a severely cold period. ‘Tochigi iW1 Go’ leaflet is a bit larger and roundish, and is colored green which is a bit paler than that of ‘Tochiotome’. ‘Tochigi iW1 Go’ petiole length is similar to that of ‘Tochiotome’. ‘Tochigi iW1 Go’ flower cluster is a straight-branch type, footstalk length is similar to that of ‘Tochiotome’, and the number of bearing of flowers at primary flower cluster is small.

Growth habit of ‘Tochigi iW1 Go’ is spreading. Lamina length, leaf width, and petiole length of ‘Tochigi iW1 Go’ are each similar to those of ‘Tochiotome’, and vigor is similar to that of ‘Tochiotome’, too. Flowering bud differentiation period is similar to or a bit earlier than that of ‘Tochiotome’, and time of beginning of flowering and time of beginning of harvesting are similar to or a bit later than those of ‘Tochiotome’. The number of bearing of flowers at primary flower cluster is smaller than that of ‘Tochiotome’.

The number of production of runners by ‘Tochigi iW1 Go’ is a bit smaller than that of ‘Tochiotome’, and rooting is slow. Flowering bud differentiation period comes a bit earlier than that of ‘Tochiotome’. Response to a treatment for promoting flowering bud differentiation by low temperature and short-day is similar to that of ‘Tochiotome’, and differentiation is accelerated. Time of beginning of flowering and time of beginning of harvesting are similar to or a bit slower than those of ‘Tochiotome’. No resistance to diseases and pests has been found so far. The number of days required for fruit to ripen from flowering to harvesting is similar to that of ‘Tochiotome’.

Glossiness of ‘Tochigi iW1 Go’ fruit skin is as excellent as that of ‘Tochiotome’. ‘Tochigi iW1 Go’ seeds are moderately below surface and are colored reddish. Time suitable for harvesting can be determined based on the degree of coloring of seeds on a sun-lit surface. ‘Tochigi iW1 Go’ fruit pulp is colored white, has a minute hollow, and does not have a characteristic fragrance. ‘Tochigi iW1 Go’ fruit shape is conical at primary flower cluster and primary axillary flower cluster, and is conical to long conical at secondary axillary flower cluster and subsequent flower clusters. Most of malformed fruits have a cylindrical shape and an ovoid shape. In and after severely cold period, deformed fruits with disordered tips (non-fertilized fruit and blue-tipped fruit) are likely to appear. Sugar content is 9.5 to 10.3 degrees, which is similar to that of ‘Tochiotome’. ‘Tochigi iW1 Go’ fruit acidity is 0.49 to 0.54%, which is lower than that of ‘Tochiotome’. ‘Tochigi iW1 Go’ fruit sugar-acid ratio is 18.8 to 20.1, which is higher than that of ‘Tochiotome’. These indicate good eating quality. Firmness of fruit is 48-60 gf/φ2 mm, which is similar to or a bit softer than that of ‘Tochiotome’.

Average weight per ‘Tochigi iW1 Go’ fruit is approximately 20 g, which is larger by 20% than that of ‘Tochiotome’, and a ratio of appearance of fruits with 22 g/fruit or more is as high as nearly 30%. Total yield is larger by 20% than that of ‘Tochiotome’. Fruit length:width ratio of ‘Tochigi iW1 Go’ is 1.2:1, and the average number of fruits per plant is 10. The fruit of ‘Tochigi iW1 Go’ has little to no hollow center. Productivity density of the fruit is 81.5 g/100 mL, which is equal to approximately 63.3 pounds per bushel.

In the case of cell nursing, the yield of salable fruit by ‘Tochigi iW1 Go’ is higher by approximately 20% than that of ‘Tochiotome’. In the case of night cooling nursing, the yield of salable fruit is equal to or higher than that of ‘Tochiotome’. In either of the tests and in either of the nursing methods, the weight per fruit is as large as approximately 20 g or more. As for the yield per flower cluster, the yield of the primary flower cluster and subsequent flower clusters is superior to that of ‘Tochiotome’. As for the ratio of the number of fruit by class of fruit weight, the ratio of fruit having a weight of 22 g or more is higher than that of ‘Tochiotome.’

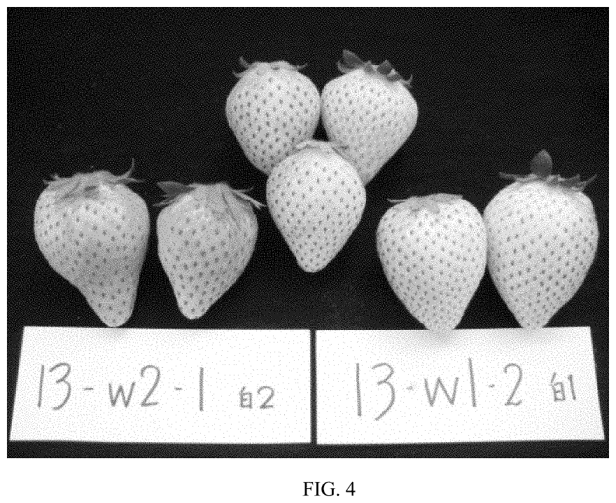

A comparison between ‘Tochigi iW1 Go’ and its parental variety ‘Wadahatsukoi’ is provided in Table 3. Fruit of ‘Wadahatsukoi’ are shown in FIG. 4.

TABLE 3
Description of physical differences between parental plants

| | | State of | |
|---|---|---|---|
| | Description of | Expression in | State of Expression in |
| Mother Variety | Characteristics | Mother Variety | ‘Tochigi iW1 Go’ |
| WADAHATSUKOI | Fruit: size | medium to large | large to very large |
| WADAHATSUKOI | Fruit (fruit skin): color | pinkish white | whitish yellow |
| 09-52-1 | Plant: growth habitat | Unknown | Spreading |
| 09-52-1 | Fruit: size | Small | Large to very large |
| 09-52-1 | Fruit (fruit skin): color | Pinkish white | Whitish yellow |
| 09-52-1 | Fruit: color of flesh | Unknown | White |
| 09-52-1 | Fruit: color of core | Unknown | White |

‘Tochigi iW1 Go’ fruit skin color is yellowish-white, which is whiter than peach white color of ‘Wadahatsukoi’. ‘Tochigi iW1 Go’ fruit shape is conical. The ratio of appearance of malformed fruits is similar to that of ‘Tochiotome’, and is larger than that of ‘Wadahatsukoi’. Deformed fruits with disordered tips, such as non-fertilized fruit and blue-tipped fruit, are likely to appear. Although ‘Tochigi iW1 Go’ fruit sugar content is similar to or a bit lower than that of ‘Tochiotome’, acidity is lower and a sugar-acid ratio is higher, indicating generally good eating quality. Firmness of fruit is similar to that of ‘Tochiotome’ and is higher than that of ‘Wadahatsukoi’.

It will be understood that the average size of the plant and fruit may vary with location, season, nutrition, irrigation, etc.

What is claimed is:

1. A new and distinct Fragaria L. plant named ‘Tochigi iW1 Go’ as illustrated and described.
